(19) United States
(12) Patent Application Publication
(10) Pub. No.: US 2025/0189506 A1
(43) Pub. Date: Jun. 12, 2025

(54) APPARATUS AND METHOD FOR CALCULATING THE CHANGE OF THE CARBON STOCK IN THE TERMINAL

(71) Applicant: NATIONAL INSTITUTE OF ECOLOGY, Chungcheongnam-do (KR)

(72) Inventors: HEON MO JEONG, Gunsan-si (KR); IN YOUNG JANG, Gunsan-si (KR); SUNG RYONG KANG, Gunsan-si (KR); EUN JIN PARK, Gunsan-si (KR)

(73) Assignee: NATIONAL INSTITUTE OF ECOLOGY, Chungcheongnam-do (KR)

(21) Appl. No.: 18/627,480

(22) Filed: Apr. 5, 2024

(30) Foreign Application Priority Data
Dec. 7, 2023 (KR) 10-2023-0176230

(51) Int. Cl. G01N 33/24

(52) U.S. Cl. CPC G01N 33/24

(57) ABSTRACT

An apparatus for calculating a change of carbon stock in a terminal includes: a database; and a control unit that confirms an administrative district and a map sheet of an evaluation area to calculate a change of carbon stock of vegetation before and after forestry project, collects forest information in an evaluation area from the database, collects one of an allometric equation and a growth coefficient using the collected forest information, calculates the carbon stock of the vegetation by applying a diameter at a breast height and a tree height to the collected allometric equation when the allometric equation is collected, determines that the calculated carbon stock of the vegetation is a carbon stock for a total area of the evaluation area, and calculates the change of the carbon stock of the vegetation by considering the carbon stock for the determined total area.

### CROSS-REFERENCE TO RELATED APPLICATION

This application claims under 35 U.S.C. § 119 (a) the benefit of Korean Patent Application No. 10-2023-0176230 filed on Dec. 7, 2023, the entire contents of which are incorporated herein by reference.

### BACKGROUND

### (a) Technical Field

The present invention relates to a terminal, and more particularly, to an apparatus and method for calculating a change of carbon stock in a terminal.

### (b) Background Art

As awareness of climate change spreads around the world, interest in greenhouse gases (CO₂, CH₄, N₂O, etc.) that cause climate change is increasing. Recently, measures to reduce greenhouse gases by utilizing greenhouse gas (carbon) storage capacity within the ecosystem have become important, and accurate calculation of greenhouse gas stock in a consistent manner is encouraged internationally.

Approximately 63.2% of Korea's land mass (10,043,185 ha) is forest land, which is distributed at a much higher rate than ecosystems such as wetlands and grasslands, so a carbon storage function of a forest is very important. However, it is predicted that the carbon storage function of Korea's forests will weaken due to repeated thinning and afforestation through continuous development plans and forest management. Therefore, to evaluate the carbon storage function of the forest, it is very important to calculate the volatility of carbon stock.

However, to date, the calculation of carbon stock in a forest is often based on actual measurement data, but when the actual measurement data is insufficient, there was a problem that it is difficult to estimate carbon stock in forests.

Therefore, the need for a method to solve these problems has emerged.

### SUMMARY OF THE DISCLOSURE

An embodiment of the present invention provides an apparatus and method for calculating a change of carbon stock in a terminal capable of calculating a carbon stock in forests even if actual measurement data is lacking.

According to an embodiment of the present invention, an apparatus for calculating a change of carbon stock in a terminal includes: a database; and a control unit that confirms an administrative district and a map sheet of an evaluation area to calculate a change of carbon stock of vegetation before and after forestry project, collects forest information in an evaluation area from the database, collects one of an allometric equation and a growth coefficient using the collected forest information, calculates the carbon stock of the vegetation by applying a diameter at a breast height and a tree height to the collected allometric equation when the allometric equation is collected, determines that the calculated carbon stock of the vegetation is a carbon stock for a total area of the evaluation area when the calculated carbon stock of the vegetation is not based on a quadrat area, and calculates the change of the carbon stock of the vegetation by considering the carbon stock for the determined total area.

According to another embodiment of the present invention, a method of calculating a change of carbon stock in a terminal includes: confirming, by a control unit, an administrative district and a map sheet of an evaluation area to calculate a change of carbon stock of vegetation before and after forestry project; collecting, by the control unit, forest information in an evaluation area from the database, and collecting one of an allometric equation and a growth coefficient using the collected forest information; when the allometric equation is collected, calculating, by the control unit, the carbon stock of the vegetation by applying a diameter at a breast height and a tree height to the collected allometric equation; when the calculated carbon stock of the vegetation is not based on a quadrat area, determining, by the control unit, that the calculated carbon stock of the vegetation is a carbon stock for a total area of the evaluation area; and calculating, by the control unit, the change of the carbon stock of the vegetation by considering the carbon stock for the determined total area.

According to embodiment of the present invention, it possible to calculate the carbon stock in a forest by calculating the change of the carbon stock even if actual measurement data is lacking.

In addition, the effects obtainable or predicted by the embodiments of the present invention will be disclosed directly or implicitly in the detailed description of the embodiments of the present invention. That is, various effects predicted according to embodiments of the present invention will be disclosed in the detailed description to be described later.

### BRIEF DESCRIPTION OF THE DRAWINGS

FIG. 1 is a block diagram of a terminal according to an embodiment of the present invention.

FIG. 2 is a flow chart for calculating a change of carbon stock of vegetation in a terminal according to an embodiment of the present invention.

FIG. 3 is a flow chart for calculating a change of carbon stock of soil in a terminal according to an embodiment of the present invention.

FIG. 4 is a schematic diagram of a forest carbon cycle according to an embodiment of the present invention.

### DETAILED DESCRIPTION

After the terms used in the present specification are briefly described, the present invention will be described in detail.

General terms that are currently widely used were selected as terms used in exemplary embodiments of the present invention in consideration of functions in the present invention, but may be changed depending on the intention of those skilled in the art or a judicial precedent, the emergence of a new technique, and the like. In addition, in a specific case, the terms arbitrarily chosen by an applicant may exist. In this case, the meaning of such terms will be mentioned in detail in a corresponding description portion of the present invention. Therefore, the terms used in the present invention should be defined on the basis of the meaning of the terms and the contents throughout the present invention rather than simple names of the terms.

Since the present invention may be variously modified and have several exemplary embodiments, specific exemplary embodiments of the present invention will be illustrated in the drawings and be described in detail in the detailed description. However, it is to be understood that the present invention is not limited to specific exemplary embodiments, but includes all modifications, equivalents, and substitutions without departing from the scope and spirit of the present invention. When it is decided that a detailed description for the known art related to the present invention may obscure the gist of the present invention, the detailed description will be omitted.

The terms “first,” “second,” and the like, may be used to describe various components, but the components are not to be construed as being limited by these terms. The terms are used only to distinguish one component from another component.

Singular forms are intended to include plural forms unless the context clearly indicates otherwise. It will be further understood that terms “include” or “formed of” used in the present specification specify the presence of features, numerals, steps, operations, components, parts, or combinations thereof mentioned in the present specification, but do not preclude the presence or addition of one or more other features, numerals, steps, operations, components, parts, or combinations thereof.

In embodiments, a ‘module’ or a ‘˜er/or’ may perform at least one function or operation, and be implemented by hardware or software or be implemented by a combination of hardware and software. In addition, a plurality of ‘modules’ or a plurality of ‘˜ers/ors’ may be integrated in at least one module and be implemented by at least one processor (not illustrated) except for a ‘module’ or an ‘˜er/or’ that needs to be implemented by specific hardware.

In exemplary embodiments of the present invention, a case in which any portion is referred to as being “connected to” another portion not only includes a case in which any one portion and another portion are “directly connected to” each other, but also a case in which any one portion and another portion are “electrically connected to” each other with the other portion interposed therebetween. In addition, unless explicitly described to the contrary, “including” any component will be understood to imply the inclusion of other components rather than the exclusion of other components.

Hereinafter, exemplary embodiments of the present invention will be described in detail with reference to the accompanying drawings so that those skilled in the art to which the present invention pertains may easily practice the present invention. However, the present invention may be implemented in various different forms and is not limited to exemplary embodiments described herein. In addition, in the drawings, portions unrelated to the description will be omitted to clearly describe the present invention, and similar portions will be denoted by similar reference numerals throughout the specification.

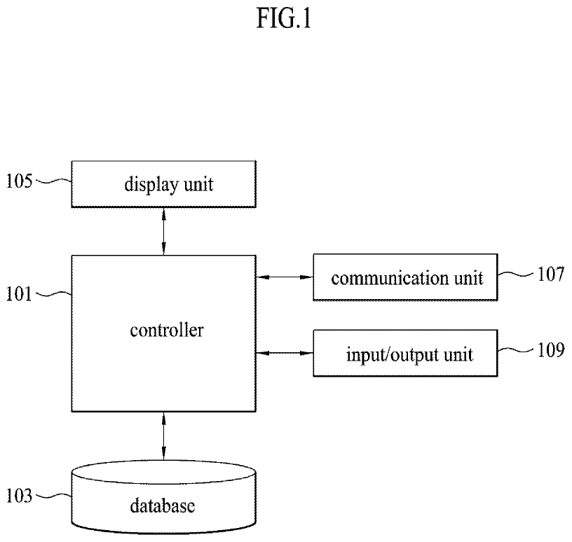

FIG. 1 is a block diagram of a terminal according to an embodiment of the present invention.

Referring to FIG. 1, the terminal includes a control unit 101, a database 103, a display unit 105, a communication unit 107, and an input/output unit 109.

Looking at each component, the database 103 stores various programs and data necessary for an operation of the terminal. For example, the database 103 may be implemented with non-volatile memory, volatile memory, flash memory, hard disk drive (HDD), solid state drive (SSD), or the like. For example, the database 103 may store a forest type map and vegetation data for each region in advance. As another example, the database 103 may store soil information actually measured for each region in advance.

The display unit 105 displays image data under the control of the control unit 101. The implementation method of the display unit 105 is not limited and may be implemented in various types of displays such as a liquid crystal display (LCD), an organic light emitting diode (OLED) display, an active-matrix organic light-emitting diode (AM-OLED), and a plasma display panel (PDP). The display unit 105 may additionally include additional components depending on its implementation method. For example, when the display unit 105 is a liquid crystal type, the display unit 105 may include an LCD display panel (not illustrated), a backlight unit (not illustrated) that supplies light to the display panel, and a panel driving substrate that drives the panel (not illustrated). The display unit 105 may be coupled to a touch panel (not illustrated) of the input/output unit 109 to be provided as a touch screen (not illustrated).

The communication unit 107 provides data transmission and reception functions of the terminal. For example, the communication unit 107 may access a server operating by national agencies under the control of the control unit 101 and request published data. The communication unit 107 may receive published data in response to the request.

The input/output unit 109 receives various instructions from the user or outputs various data to an external device. For example, the input/output unit 109 may include at least one of a keyboard, a mouse, a key, a touch panel, and a pen recognition panel.

The control unit 101 controls the overall operation of the terminal using various programs stored in the database 103.

For example, the control unit 101 may calculate a change of carbon stock before and after a forestry project. For example, the forestry project may be a project that carries out logging and afforestation through a forest tending project, etc.

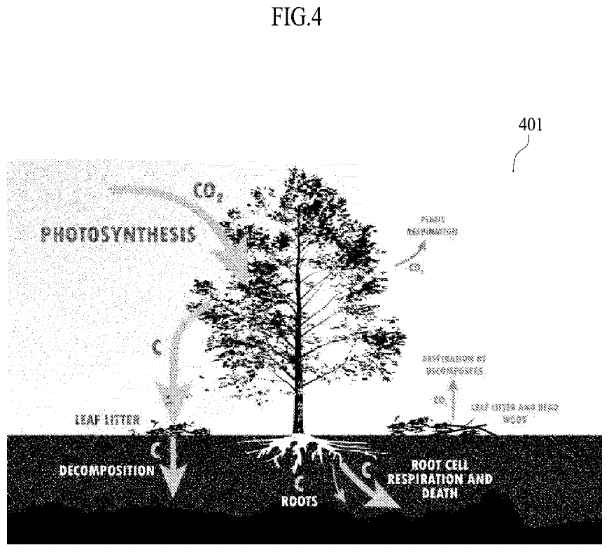

For example, carbon in forests may be stored in vegetation and soil, as illustrated at 401 in FIG. 4. Here, the carbon stock of vegetation refers to storing carbohydrates synthesized by plants through photosynthesis within the plant body, and carbon is stored in stems, branches, roots, and leaves. Vegetation that stores carbon (greenhouse gas) inside the plant enters the soil in the form of fallen leaves, fallen branches, and dead wood. In this case, the carbon is broken down into small pieces by decomposition activity of soil microorganisms and stored in the soil in the form of organic matter. Therefore, when calculating the carbon stock in the forest, the carbon stock in both vegetation and soil should be considered.

For example, the change of the carbon stock of the vegetation and soil after deforestation may be basically calculated by comparing the carbon stock the before and after the forestry project.

For example, the control unit 101 may calculate the change of the carbon stock of the vegetation.

In more detail, the control unit 101 may confirm an administrative district and map sheet of the evaluation area for evaluating the carbon stock in the vegetation. These operations are performed to confirm and collect the forest type level of the evaluation area or to confirm available vegetation data (diameter at breast height (DBH), tree height, etc.).

In addition, the control unit 101 may collect forest type map or vegetation data using the confirmed administrative district and map sheet, and collect forest information in the evaluation area based on the collected forest type map or vegetation data. For example, the forest information includes the forest type map information including forest type, age class, DBH class, and density, as well as vegetation (diameter at breast height (hereinafter referred to as ‘DBH’) and tree height) data collected from actual field surveys, related organizations, reports, etc. For example, the forestry information may be found on the forest type map. Meanwhile, in this operation, when either the forest type map information or vegetation data is collected, the carbon stock in the forest may be calculated.

Additionally, the control unit 101 may collect an allometric equation or growth coefficient based on the collected forest type map information or vegetation data.

For example, the growth coefficient is an index of a volume of trees and may represent the quantification of the relationship between the forest type, age class, DBH class, and density of the forest type map and the volume. For example, the growth coefficient may use the research results of Kim So-won et al (2014).

For example, the allometric equation is a relation equation that estimates the carbon stock in the entire tree or each organ (stem, branches, leaves, roots, etc.) by using the tree's biological data such as the diameter at breast height and tree height), Here, various types of equations may exist depending on species of trees, type of leaf (coniferous tree, broad-leaved tree), etc.

For example, the allometric equation may be represented by Equation 1.

$$\begin{matrix}log W = a log DBH + b \\ log W = a log {D}^{2} H + b \\ W = a ( DBH ) b \\ W = a ( {D}^{2} H ) b\end{matrix} \qquad [ Equation 1 ]$$

For example, W may denote the carbon stock, DBH may denote the diameter at breast height, H may denote the tree height, and a and b may denote the constants.

The control unit 101 may confirm whether the allometric equation has been collected. As a result of confirmation, when the allometric equation is collected, the control unit 101 may calculate the carbon stock of the vegetation by applying the diameter at breast height and tree height to the allometric equation.

For example, the carbon stock calculation equation using the growth coefficient may be represented by Equation 2.

$$CarbonStock = V \times BEF \times WD \times C \qquad [ Equation 2 ]$$

For example, V may denote the volume (growth coefficient), biomass expansion factor (BEF) may denote biological expansion coefficient, wood density (WD) may denote wood basic density, and carbon fraction (C) may denote carbon conversion coefficient.

In this case, the control unit 101 may confirm whether the calculated carbon stock of the vegetation is based on a dry weight. As a result of confirmation, if the calculated carbon stock of the vegetation is based on the dry weight, the control unit 101 may recalculate the carbon stock of the vegetation by applying the carbon conversion coefficient to the calculated carbon stock of the vegetation.

On the other hand, when the allometric equation is not collected, the control unit 101 may calculate the carbon stock of the vegetation by applying the forest type map information and growth coefficient to the calculation equation.

The control unit 101 may confirm whether the calculated carbon stock of the vegetation is based on a quadrat area.

As a result of confirmation, when the calculated carbon stock of the vegetation is based on the quadrat area, the control unit 101 may calculate carbon stock per unit area using the calculated carbon stock of the vegetation, and uses the calculated carbon stock per unit area to convert the carbon stock of the total area.

On the other hand, if the calculated carbon stock of the vegetation is not based on the quadrat area, the control unit 101 may determine that the calculated carbon stock of the vegetation is the carbon stock of the total area.

In addition, the control unit 101 may calculate the change of the carbon stock of the vegetation before and after the forestry project by considering the carbon stock of the total area. For example, the control unit 101 may calculate the carbon stock of the vegetation before the forestry project and the carbon stock of the vegetation after the forestry project using the carbon stock of the total area. The control unit 101 may compare the carbon stock of the vegetation before the forestry project with the carbon stock of the vegetation after the forestry project to calculate the change of the carbon stock of the vegetation before and after the forestry project, which is the difference between the carbon stock of the vegetation before and after the forestry project.

As another example, the control unit 101 may calculate the change of the carbon stock of the soil.

Describing in more detail, the control unit 101 may confirm the administrative district of the evaluation area. The control unit 101 may collect the soil information in the evaluation area. For example, the soil information may include soil thickness, soil bulk density, organic carbon content and small rock content in the soil.

Meanwhile, in order to accurately derive the carbon stock of the soil in the evaluation area, the soil information in the evaluation area should be collected using actual measurement information in the target area. For example, the actual measurement information may represent information generated by actually measuring the soil in the evaluation area. Therefore, the control unit 101 may confirm whether it is possible to obtain the soil information in the evaluation area as the actual measurement information in the evaluation area.

As a result of confirmation, when the soil information in the evaluation area is obtained as the actual measurement information, the control unit 101 may collect the soil information in the evaluation area based on the actual measurement information.

On the other hand, when the soil information in the evaluation area is not obtained as the actual measurement information, the control unit 101 may acquire data (for example, forest site soil map and soil information system) published by national agencies and previous research data, and collect the soil information in the evaluation area based on the published data and previous research data obtained.

In other words, since it is difficult to obtain the actual measurement information of most projects, the control unit 101 may collect the soil information in the evaluation area using data published by national agencies or data used in previous studies.

For example, the soil thickness and small rock content may be obtained using the forest site soil map (http://data.nsdi.go.kr) published by the Korea Forest Service. The organic carbon content in the soil may be obtained from the soil Information system (https://soil.rda.go.k) provided by the Rural Development Administration. In this case, since the soil information system does not directly present an organic carbon content but presents a soil organic matter content value, the organic carbon content may be used by dividing the soil organic matter content value by 1.724. Since it is not possible to obtain the soil bulk density directly through published data, the soil bulk density may be obtained using host rock information. The host rock information may be acquired from the forest site soil map. For example, inferring the soil bulk density from the acquired host rock information may use the information presented by Jeong Jin-hyeon et al., (2003).

For example, the soil bulk density may be inferred by applying the host rock information in Table 1.

TABLE 1
Soil bulk density using host rock information (Jeong Jin-hyun et al., 2003)

| | A layer | | | B layer | | |
|---|---|---|---|---|---|---|
| | Igneous | Metamorphic | Sedimentary | Igneous | Metamorphic | Sedimentary |
| | rock | rock | rock | rock | rock | rock |
| Soil bulk | 0.91 | 0.84 | 0.97 | 1.04 | 1.02 | 1.04 |
| density | | | | | | |
| (g/cm3) | | | | | | |

The control unit 101 may calculate the carbon stock of the soil using the collected soil information in the evaluation area.

For example, the equation for calculating the carbon stock of the soil may be represented by Equation 3.

$$CS ( Mg / ha ) = T ( cm ) \times BD ( g / {cm}^{2} ) \times C \times ( 1 - CF ) \qquad [ Equation 3 ]$$

For example, carbon sequestration (CS) may denote the carbon stock of the soil, thickness (T) may denote the thickness of the soil, bulk density (BD) may denote the soil bulk density, carbon content (C) may denote the organic carbon in the soil, and coarse fragment (CF) denote the small rock content.

In this case, the control unit 101 calculates the carbon stock of the soil by layer (O layer, A layer, and B layer) of each soil depth, respectively, derives the carbon stock by layer of each soil depth, and sums the derived carbon stocks to determine the carbon stock of the soil in the evaluation area.

The control unit 101 may calculate the change of the carbon stock of the soil before and after the forestry project by considering the determined carbon stock of the soil in the evaluation area. For example, the control unit 101 may calculate the carbon stock of the vegetation before the forestry project using the determined carbon stock of the soil in the evaluation area. For example, when the determined carbon stock of the soil in the evaluation area is calculated using data published by national agencies, the control unit 101 may determine that the determined carbon stock of the soil in the evaluation area is the carbon stock of the soil before the forestry project.

For example, the control unit 101 may determine the carbon stock of the soil after the forestry project by considering the determined carbon stock of the soil before the forestry project. For example, the control unit 101 may calculate the carbon stock of the soil after the forestry project by reducing the carbon stock of the soil before the forestry project determined according to the forest type of the evaluation area. For example, the control unit 101 may calculate the carbon stock of the soil after the forestry project by reducing the carbon stock of the soil before the forestry project, which was determined based on Nave et al. (2010), which presented reduction rates for each forest type. For example, referring to Nave et al. (2010), the reduction rate for coniferous and mixed forests is 20 percent, and the reduction rate for broad-leaved forests is 36 percent.

For example, the control unit 101 may calculate the change of the carbon stock of the soil before and after the forestry project, which is the difference in the carbon stock of the soil before and after the forestry project, by comparing the determined carbon stock of the soil before the forestry project with the carbon stock of the soil after the determined forestry project.

According to embodiment of the present invention, through the operation, it possible to calculate the carbon stock of the forest by calculating the change in the carbon stock even if the actual measurement data is lacking.

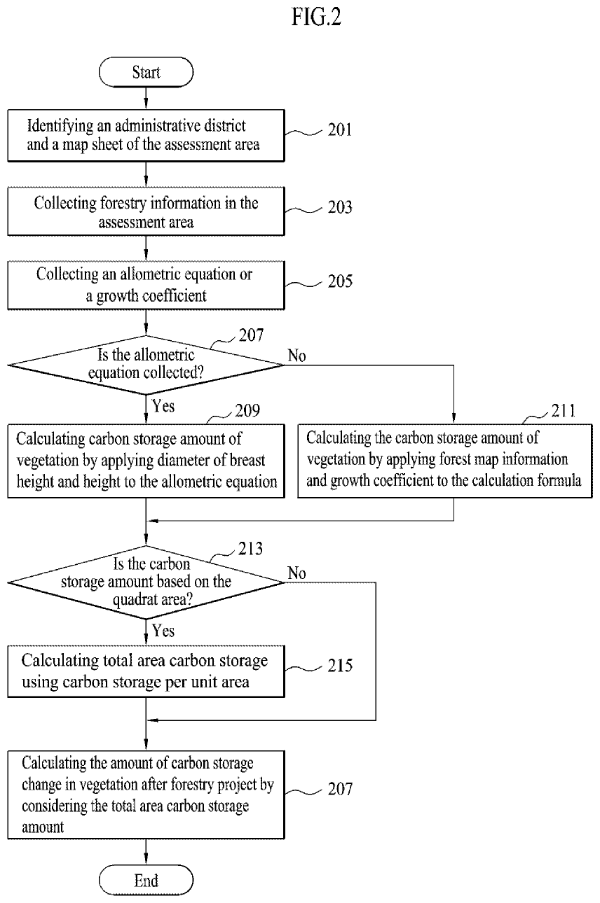

FIG. 2 is a flow chart for calculating a change of carbon stock of vegetation in a terminal according to an embodiment of the present invention.

Referring to FIG. 2, the control unit 101 of the terminal confirms an administrative district and map sheet of an evaluation area for evaluating carbon stock of vegetation in step 201 and then proceeds to step 203.

Meanwhile, step S201 is performed to confirm and collect a forest type map of the evaluation area or confirm available vegetation data (diameter at breast height (DBH), tree height, etc.).

In step S203, the control unit 101 collects the forest type map or vegetation data using the confirmed administrative district and map sheet, collects the forest information in the evaluation area based on the collected forest type map or vegetation data, and then proceeds to step S205. For example, the forestry information may be found on the forest type map.

Meanwhile, in step S203, when either the forest map information or vegetation data is collected, the carbon stock in the forest may be calculated.

The control unit 101 may collect the allometric equation or growth coefficient based on the collected forest map information or vegetation data in step 205, and then proceeds to step 207. For example, the allometric equation may be represented by Equation 1.

In step 207, the control unit 101 confirms whether the allometric equation has been collected.

As a result of the confirmation, when the allometric equation is collected, the control unit 101 proceeds to step 209. If not, the control unit 101 determines that the growth coefficient has been collected and proceeds to step 211.

If proceeding to step 209, the control unit 101 calculates the carbon stock of the vegetation by applying the diameter at breast height and tree height to the allometric equation and then proceeds to step 213. For example, the carbon stock calculation equation using the growth coefficient may be represented by Equation 2.

In this case, the control unit 101 confirms whether the calculated carbon stock of the vegetation is based on the dry weight. As a result of the confirmation, when the calculated carbon stock of the vegetation is based on the dry weight, the control unit 101 recalculates the carbon stock of the vegetation by applying the carbon conversion coefficient to the calculated carbon stock of the vegetation and proceeds to step 213. On the other hand, when the calculated carbon stock of the vegetation is not based on the dry weight, the control unit 101 proceeds directly to step 213.

In step 211, the control unit 101 calculates the carbon stock of the vegetation by applying the forest type map information and growth coefficient to the calculation formula, and then proceeds to step 213.

In step 213, the control unit 101 confirms whether the calculated carbon stock of the vegetation is based on the quadrat area.

As a result of the confirmation, when the calculated carbon stock of the vegetation is based on the quadrat area, the control unit 101 proceeds to step 215. If not, the control unit 101 determines that the calculated carbon stock of the vegetation is the entire target area and then proceeds to step 217.

If proceeding to step 215, the control unit 101 calculates the carbon stock per unit area using the calculated carbon stock of the vegetation, converts the carbon stock of the total area using the calculated carbon stock per unit area, and then proceeds to step 217.

In step 217, the control unit 101 calculates the change of the carbon stock of the vegetation before and after the forestry project by considering the carbon stock of the total area. For example, the control unit 101 may calculate the carbon stock of the vegetation before the forestry project and the carbon stock of the vegetation after the forestry project using the carbon stock of the total area. The control unit 101 may compare the carbon stock of the vegetation before the forestry project with the carbon stock of the vegetation after the forestry project to calculate the change of the carbon stock of the vegetation before and after the forestry project, which is the difference between the carbon stock of the vegetation before and after the forestry project.

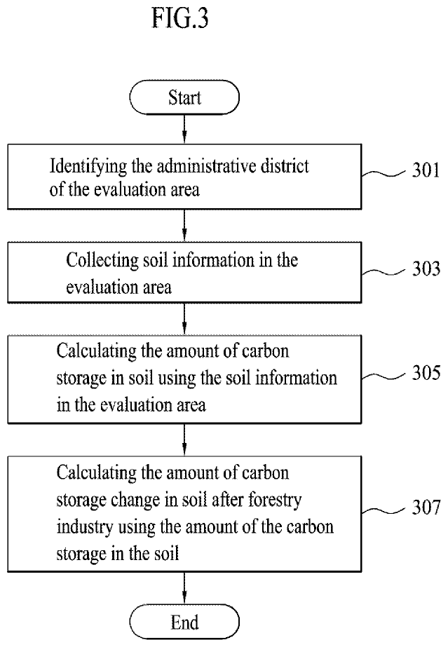

FIG. 3 is a flowchart for calculating the change of carbon stock of soil in a terminal according to an embodiment of the present invention.

Referring to FIG. 3, the control unit 101 of the terminal confirms the administrative district of the evaluation area in step 301 and then proceeds to step 303.

In step 303, the control unit 101 collects the soil information in the evaluation area and then proceeds to step 305.

Meanwhile, in order to accurately derive the carbon stock of the soil in the evaluation area, the soil information in the evaluation area should be collected using the actual measurement information in the target area. Therefore, the control unit 101 confirms whether it is possible to obtain the soil information in the evaluation area as actual measurement information in the evaluation area.

As a result of the confirmation, when the soil information in the evaluation area is obtained as the actual measurement information, the control unit 101 collects the soil information in the evaluation area based on the actual measurement information. On the other hand, when the soil information in the evaluation area is not obtained as the actual measurement information, the control unit 101 acquires data (for example, forest site soil map and soil information system) published by national agencies and previous research data, and collects soil information in the evaluation area based on the published data and previous research data acquired.

In other words, since it is difficult to obtain the actual measurement information in most projects, the control unit 101 collects the soil information in the evaluation area using data published by national agencies or data used in previous studies.

In step 305, the control unit 101 calculates the carbon stock of the soil using the collected soil information in the evaluation area and then proceeds to step 307.

For example, the equation for calculating the carbon stock of the soil may be represented by Equation 3.

Meanwhile, the control unit 101 calculates the carbon stock of the soil by layer (O layer, A layer, and B layer) of each soil depth, derives the carbon stock by layer of each soil depth, and sums the derived carbon stocks to determine the carbon stock of the soil in the evaluation area.

In step 307, the control unit 101 calculates the change of carbon stock of soil before and after the forestry project by considering the determined carbon stock of the soil in the evaluation area. For example, the control unit 101 may calculate the carbon stock of the vegetation before the forestry project using the determined carbon stock of the soil in the evaluation area. For example, when the determined carbon stock of the soil in the evaluation area is calculated using the data published by national agencies, the control unit 101 may determine that the determined carbon stock of the soil in the evaluation area is the carbon stock of the soil before the forestry project.

For example, the control unit 101 may determine the carbon stock of the soil after the forestry project by considering the determined carbon stock of the soil before the forestry project. For example, the control unit 101 may calculate the carbon stock of the soil after the forestry project by reducing the determined carbon stock of the soil before the forestry project according to the forest type of the evaluation area. For example, the control unit 101 may calculate the carbon stock of the soil after the forestry project by reducing the carbon stock of the soil before the forestry project, which was determined based on Nave et al. (2010), which presented the reduction rates for each forest type. For example, referring to Nave et al., (2010), the reduction rate for coniferous and mixed forests may be 20 percent, and the reduction rate for broad-leaved forests may be 36 percent.

For example, the control unit 101 may calculate the change of the carbon stock of the soil before and after the forestry project, which is the difference in the carbon store of the soil before and after the forestry project, by comparing the determined carbon stock of the soil before the forestry project with the determined carbon stock of the soil after the forestry project.

According to an embodiment of the present invention, through this process, it is possible to calculate the change of the carbon stock and calculate the carbon stock of the forest even if the actual measurement data is lacking.

According to embodiment of the present invention, it possible to calculate the carbon stock in a forest by calculating the change of the carbon stock even if actual measurement data is lacking.

In addition, the effects obtainable or predicted by the embodiments of the present invention will be disclosed directly or implicitly in the detailed description of the embodiments of the present invention. That is, various effects predicted according to embodiments of the present invention will be disclosed in the detailed description to be described later.

Although exemplary embodiments of the present invention have been illustrated and described, the present invention is not limited to the above-mentioned specific exemplary embodiment, but may be variously modified by those skilled in the art to which the present invention pertains without departing from the spirit and scope of the present invention as claimed in the claims. In addition, such modifications should also be understood to fall within the scope of the present invention.

1. An apparatus for calculating a change of carbon stock in a terminal, comprising:
a database; and
a control unit that confirms an administrative district and a map sheet of an evaluation area to calculate a change of carbon stock of vegetation before and after forestry project, collects forest information in an evaluation area from the database, collects one of an allometric equation and a growth coefficient using the collected forest information, calculates the carbon stock of the vegetation by applying a diameter at a breast height and a tree height to the collected allometric equation when the allometric equation is collected, determines that the calculated carbon stock of the vegetation is a carbon stock for a total area of the evaluation area when the calculated carbon stock of the vegetation is not based on a quadrat area, and calculates the change of the carbon stock of the vegetation by considering the carbon stock for the determined total area.

2. The apparatus of claim 1, wherein the control unit calculates the carbon stock of the vegetation by applying a forest type map information and the collected growth coefficient to a calculation equation when the growth coefficient is collected.

3. The apparatus of claim 1, wherein the control unit determines that the calculated carbon stock of the vegetation is carbon stock per unit area of the evaluation area, calculates the carbon stock for the total area using the carbon stock per unit area when the calculated carbon stock of the vegetation is based on the quadrat area, and calculates the change of the carbon stock of the vegetation by considering the carbon stock for the calculated total area.

4. The apparatus of claim 1, wherein the control unit confirms the administrative district of the evaluation area to calculate a change of carbon stock of soil before and after the forestry project, collects soil information in the evaluation area from the database, calculates the carbon stock of the soil using the collected soil information, and calculates the change of the carbon stock of the soil calculated using the carbon stock of the soil.

5. The apparatus of claim 4, wherein the control unit determines that the carbon stock of the soil is the carbon stock of the soil before the forestry project when the soil information is collected through data published by national agencies and prior research data, derives the carbon stock of the soil after the forestry project using the determined carbon stock of the soil based on a forest type in the evaluation area, and compares the determined carbon stock of the soil with the derived carbon stock of the soil to calculate the change of the carbon stock of the soil.

6. A method of calculating a change of carbon stock in a terminal, comprising:
confirming, by a control unit, an administrative district and a map sheet of an evaluation area to calculate a change of carbon stock of vegetation before and after forestry project;
collecting, by the control unit, forest information in an evaluation area from the database, and collecting one of an allometric equation and a growth coefficient using the collected forest information;
calculating, by the control unit, the carbon stock of the vegetation by applying a diameter at a breast height and a tree height to the collected allometric equation when the allometric equation is collected;
determining, by the control unit, that the calculated carbon stock of the vegetation is a carbon stock for a total area of the evaluation area when the calculated carbon stock of the vegetation is not based on a quadrat area; and
calculating, by the control unit, the change of the carbon stock of the vegetation by considering the carbon stock for the determined total area.

7. The method of claim 6, further comprising:
calculating, by the control unit, the carbon stock of the vegetation by applying a forest type map information and the collected growth coefficient to a calculation equation when the growth coefficient is collected.

8. The method of claim 6, further comprising:
determining, by the control unit, that the calculated carbon stock of the vegetation is a carbon stock for a unit area of the evaluation area when the calculated carbon stock of the vegetation is based on a quadrat area; and
calculating, by the control unit, carbon stock for the total area using the carbon stock per unit area, and calculating the change of the carbon stock of the vegetation by considering the calculated carbon stock for the total area.

9. The method of claim 6, further comprising:
confirming, by the control unit, the administrative district of the evaluation area to calculate a change of carbon stock of soil before and after the forestry project;
collecting, by the control unit, soil information in the evaluation area from the database;
calculating, by the control unit, the carbon stock of the soil using the collected soil information; and
calculating, by the control unit, the change of the carbon stock of the soil calculated using the carbon stock of the soil.

10. The method of claim 9, wherein the calculating of the change of the carbon stock of the soil includes determining that the carbon stock of the soil is the carbon stock of the soil before the forestry project when the soil information is collected through data published by national agencies and prior research data,
deriving the carbon stock of the soil after the forestry project using the determined carbon stock of the soil based on a forest type in the evaluation area, and
comparing the determined carbon stock of the soil with the derived carbon stock of the soil to calculate the change of the carbon stock of the soil.
